(19) United States
(12) Patent Application Publication
(10) Pub. No.: US 2019/0240250 A1
(43) Pub. Date: Aug. 8, 2019

(54) Heparin and Statin Combinations for Preventing Metastatic Cancer

(71) Applicant: University College Dublin, National University of Ireland, Dublin, Dublin (IE)

(72) Inventors: Fionnuala Ni Ainle, Dublin (IE); Barry Kevane, Dublin (IE)

(21) Appl. No.: 16/339,341

(22) PCT Filed: Sep. 20, 2017
(86) PCT No.: PCT/EP2017/073837

§ 371 Date: Apr. 3, 2019

(30) Foreign Application Priority Data
Oct. 4, 2016 (GB) 1616853.6

(51) Int. Cl. A61K 31/727; A61K 31/366; A61K 9/00

(52) U.S. Cl. CPC A61K 31/727; A61K 9/0053; A61K 31/366

(57) ABSTRACT

A combination of drugs for use in a method for the prevention or treatment of cancer metastasis in an individual with a primary cancer is provided. The combination is administered to the individual and comprises a sub-anticoagulating heparin formulation, for example a sub-anticoagulating dose of a low molecular weight heparin (LMWH) formulation, and a therapeutically effective dose of a statin. The combination significantly decreases endothelial barrier permeability while avoiding risk of bleeding by using a sub-anticoagulation heparin formulation.

### FIELD OF THE INVENTION

The present invention relates to methods of preventing metastatic cancer disease in an individual. Also contemplated are therapeutic compositions for preventing development of metastatic cancer disease in an individual.

### BACKGROUND TO THE INVENTION

Complications arising as a result of the growth of metastatic tumours account for the vast majority of cancer associated deaths. Consequently, therapies which are directed at interrupting metastasis would be predicted to improve patient outcomes. Metastasis is a complex, multi-step process involving the detachment of malignant cells from the primary tumour bulk and their migration through the systemic circulation to a distant target organ, where they invade and proliferate to form secondary tumour deposits. In vitro and in vivo studies of human metastasis suggest that the key determinants of successful completion of cancer dissemination are the ability of circulating tumour cells to extravasate through the vascular endothelium at their target site and their ability to withstand the hostile conditions of the foreign micro-environment. In support of this hypothesis, reports from studies which have explored the phenomenon of organ-specific tropism in the setting of metastasis (the marked predilection towards the colonization of specific tissues or organs exhibited by malignant tumours) have demonstrated that tumours induce abnormal permeability in the normally impermeable vascular endothelium of their metastatic targets and that this effect appears to be crucial to successful tumour invasion. Moreover, the findings of these studies suggest that inhibition of tumour-induced vascular permeability effectively inhibits the emergence of metastatic tumours, highlighting the phenomenon of tumour-induced endothelial permeability as a promising target for anti-metastatic therapy.

The low molecular weight heparins (LMWH) are a class of anticoagulant drug derived by depolymerisation of unfractionated heparin (UFH), a naturally occurring polymer of heparin polysaccharide chains extracted from bovine or porcine mucosa. The LMWHs are similar in structure to the endogenous heparan sulphate proteoglycans (HSPG) which are widely expressed in the extracellular matrix of human tissues and on the cell surface of all eukaryotic cells. These HSPGs exhibit marked structural and functional diversity and play key roles in the regulation of numerous physiological and pathological processes including in regulating the cell signaling activity of numerous agonists (such as the HSPG-mediated co-factor activity implicated in VEGF and thrombin cell signaling), acting as sites of sequestration of various growth factors (such as members of the fibroblast growth factor family of signaling proteins) and as regulators of the composition of the ECM. Commercial formulations of LMWH are currently exclusively used for the treatment and prevention of thrombosis however several studies of LMWH in both in vitro and in vivo models of metastasis suggest that these heparin polysaccharides also appear to influence cell signaling pathways implicated in tumour dissemination.

The findings in these pre-clinical studies which suggest that LMWH exerts anti-metastatic properties have been reflected in several clinical trials which have suggested that LMWH improves survival (independent of the reduction in thrombotic risk) among patients with cancer with early-stage disease (prior to the emergence of established metastatic tumours). However, uncertainty remains as to the magnitude of this survival benefit and as to the specific sub-groups of cancer patients who would be likely to derive an additional survival benefit from LMWH exposure.

As a result of this uncertainty and in view of the significant risk of major haemorrhage associated with anticoagulant therapy, current clinical practice guidelines recommend against the use of LMWH in cancer except for the treatment or prevention of thrombosis.

Non-anticoagulant heparin has also been suggested in the literature as having an anti-metastatic effect (Sudha et al. Clin. Exp. Metastasis (2012), 29: 431-439; Mouse et al. Thromb. Haemost. (2006) 96(6), 816-821; Duckworth et al. Oncotarget (2015), Vol 6, No. 27); WO2013/045955 and US2015/132399. As this is a modified form of heparin, it is more expensive than LMWH and would have greater regulatory challenges compared with use of conventional LMWH.

Statins are also recognised in the literature as having anti-metastatic effects (Wolfe et al. Breast Cancer Res. Treat. (2015) 154, 495-508; Mandal et al. Journal of Biological Chemistry (2011) Vol. 286 No. 13); Salis et al. Tumour Biol. (2016) 37, 3017-3024; and Fang et al. PLOS ONE (2013), Vol. 8, Issue 5.

It is an object of the invention to overcome at least one of the above-referenced problems.

### SUMMARY OF THE INVENTION

The use of effective levels of heparin as a prophylactic medicine to inhibit metastatic cancer disease in patients with primary cancer by lowering endothelial barrier permeability has been inhibited by the risk of bleeds in the treated patients. The present invention is based on the finding that a sub-anticoagulation heparin formulation, for example a sub-anticoagulating dose of low molecular weight heparin (LMWH), can be employed to inhibit metastasis when it is administered in combination with a statin. The in-vitro data below demonstrates significant lowering of endothelial barrier permeability across a range of sub-anticoagulating doses of LMHW when the therapy includes administration of a statin. In particular, a statin on its own decreases thrombin induced permeability to 77% to 50% across a range of clinically relevant statin concentrations (5-20 nM) (FIG. 5A—continuous line), whereas co-administration with a sub-anticoagulating dose of Tinzaparin (LMWH) of 0.1 IU/ml decreases thrombin induced permeability to 62% to 10% across the same range of clinically relevant statin concentrations (5-20 nM) (FIG. 5A—broken line). In particular, combining a sub-anticoagulating dose of LMHW with statins in the 15-20 nM range causes a decrease in thrombin induced permeability to 10-25% (FIG. 5A—broken line). Similarly, synergistic effects are obtained across a range of non-anticoagulating LMWH concentrations (0.1 to 0.2 IU/ml) where co-administration with a clinically relevant concentration of a statin reduces the thrombin induced permeability from 99-75% (no statin—FIG. 1(D) right panel) to 55-48% (with statin—FIG. 5(B)). The present invention therefore realises a method of successfully employing a heparin formulation such as LMWH as an anti-metastatic agent without the risk of bleeding in treated patients.

According to a first aspect of the present invention, there is provided a combination of drugs for use in a method for the prevention or treatment of cancer metastasis in an individual (typically an individual with a primary cancer), in which the combination is administered to the individual and comprises a sub-anticoagulating heparin formulation and a therapeutically effective dose of a statin.

In one embodiment, the sub-anticoagulating heparin formulation is selected from LMWH provided at a sub-anticoagulating dose.

In one embodiment, the sub-anticoagulating heparin formulation is selected from a low molecular weight fraction of LMWH.

In one embodiment, the sub-anticoagulating heparin formulation is selected from a non-anticoagulating heparin formulation.

In one embodiment, the sub-anticoagulating dose of LMWH is sufficient to achieve a plasma concentration of LMWH of less than 0.25 IU/ml. In one embodiment, the sub-anti-coagulating dose of LMWH is sufficient to achieve a plasma concentration of LMWH of less than 0.05 to 0.2 IU/ml.

In one embodiment, the therapeutically effective dose of statin is sufficient to achieve a plasma concentration of statin of 5-30 nM.

In one embodiment, the sub-anticoagulating heparin formulation is administered parenterally and the statin is administered orally.

In one embodiment, the sub-anticoagulating heparin formulation is administered after administration of the statin.

In one embodiment, the primary cancer is an early stage cancer, for example a stage I, II or III cancer.

In one embodiment, the individual does not have elevated plasma cholesterol levels.

Also contemplated are pharmaceutical compositions comprising a sub-anticoagulating heparin formulation and a statin. In one embodiment, the sub-anticoagulating heparin formulation comprises a sub-coagulating dose of LMWH. In one embodiment, the LMWH is a low molecular weight fraction of LMWH.

In one embodiment, the pharmaceutical composition of the invention takes the form of a liquid suitable for parenteral administration to an individual. In one embodiment, the composition comprises a sub-anticoagulating dose of a low molecular weight heparin (LMWH) formulation and a therapeutically effective dose of a statin. The pharmaceutical formulations can also be in the form of a liquid, a powder, a tablet, a capsule, a soft chew, or a gel. Generally, the pharmaceutical formulation contains one or more pharmaceutically acceptable carriers, i.e., a carrier that it is typically compatible with the active ingredients of the composition, and preferably, capable of stabilizing the active ingredients and not deleterious to the subject to be treated.

Also contemplated is a kit of parts comprising one or more doses of a sub-anticoagulating heparin formulation and one or more therapeutically effective doses of a statin.

In one embodiment, the sub-anticoagulating heparin formulation is a sub-coagulating dose of LMWH. In one embodiment, the heparin formulation is formulated as a liquid suitable for parenteral administration and the statin is formulated for oral administration.

An alternative, but linked solution to the problem of using LMWH for the treatment of metastasis, involves using a form of heparin that is depleted in antithrombin-binding pentasaccharide (A-domain). Fractions of heparin and LMWH of low molecular weight (i.e. a mean molecular weight of less than 5 or 4 KDa) are known to have reduced anticoagulant activity. The Applicants have discovered that such heparin fractions retain the ability to reduce endothelial barrier permeability. In addition, heparin (fractionated or unfractionated) can be treated for depletion of antithrombin-binding pentasaccharide (A-domain).

Thus, in another aspect the invention provides a heparin formulation depleted in antithrombin-binding pentasaccharide (A-domain), for use in a method for the prevention or treatment of cancer metastasis in an individual (typically an individual with a primary cancer). Examples of such heparin formulations include low molecular weight fractions of LMWH which can be prepared using conventional technology, such as size exclusion chromatography, to achieve the desired fraction. Methods of preparing the low molecular weight fractions are described below and are described in the literature. Other methods of preparing antithrombin-depleted heparin formulations are described below.

In one embodiment, the low molecular weight fraction has a mean molecular weight of less than 5 KDa. In one embodiment, the low molecular weight fraction has a mean molecular weight of less than 4 KDa. In one embodiment, the low molecular weight fraction has a mean molecular weight of less than 3 KDa. In one embodiment, the low molecular weight fraction has a mean molecular weight of about 2-4 KDa. In one embodiment, the low molecular weight fraction has a mean molecular weight of about 2-3 KDa.

In another aspect, the invention provides the use as a medicament of a low molecular weight fraction of LMWH.

Other aspects and preferred embodiments of the invention are defined and described in the other claims set out below.

### BRIEF DESCRIPTION OF THE FIGURES

FIG. 1: LMWH supports endothelial barrier function and attenuates agonist-induced endothelial permeability

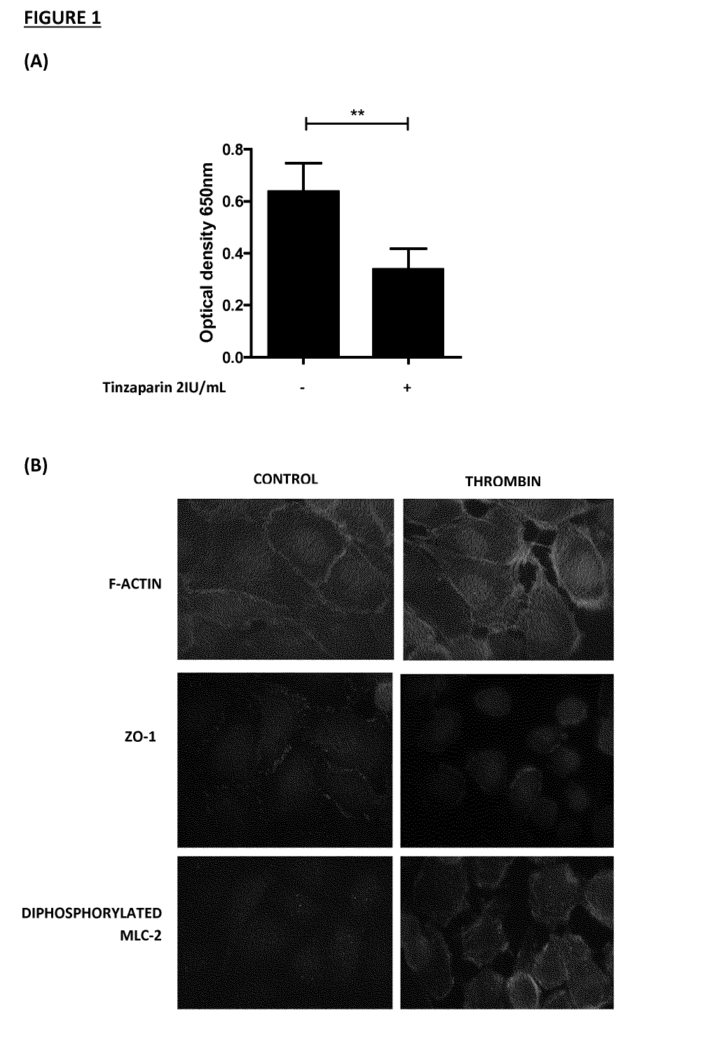

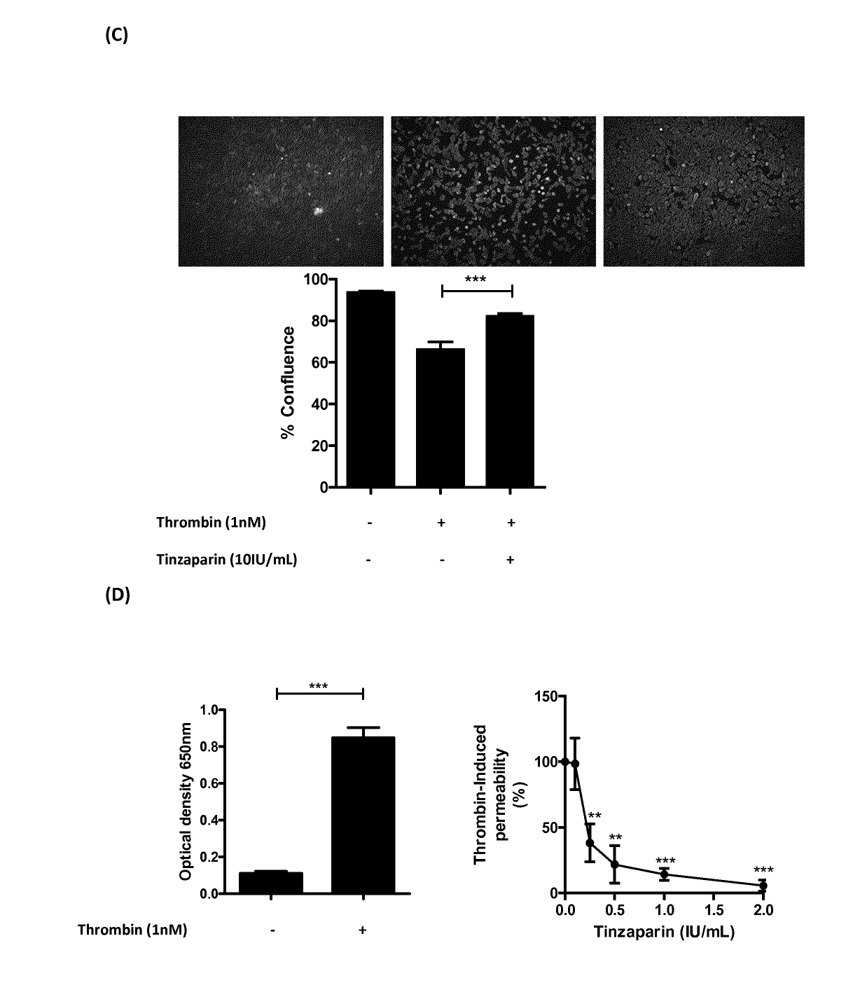

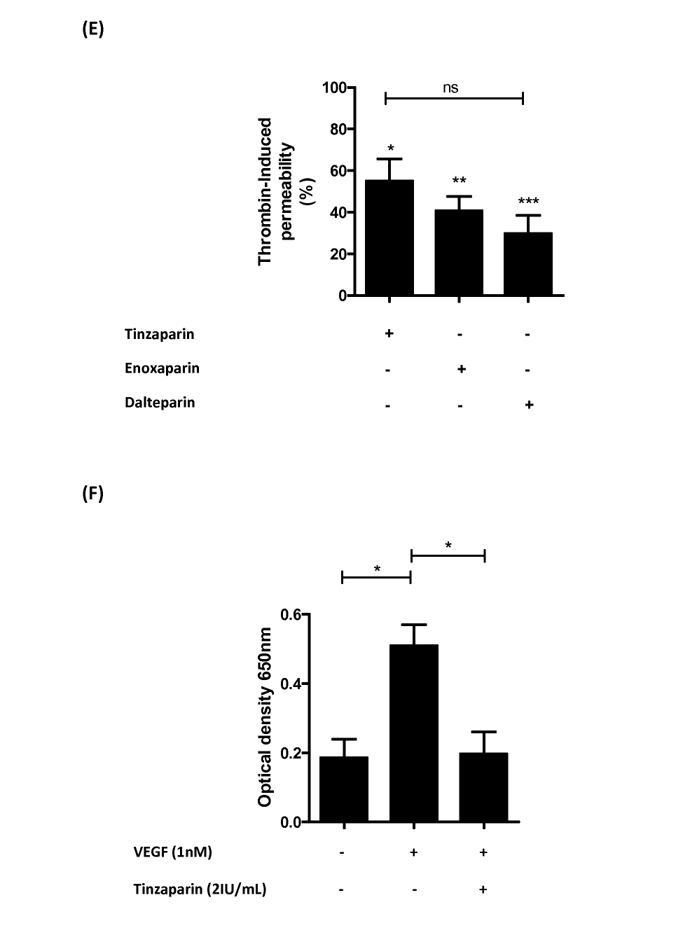

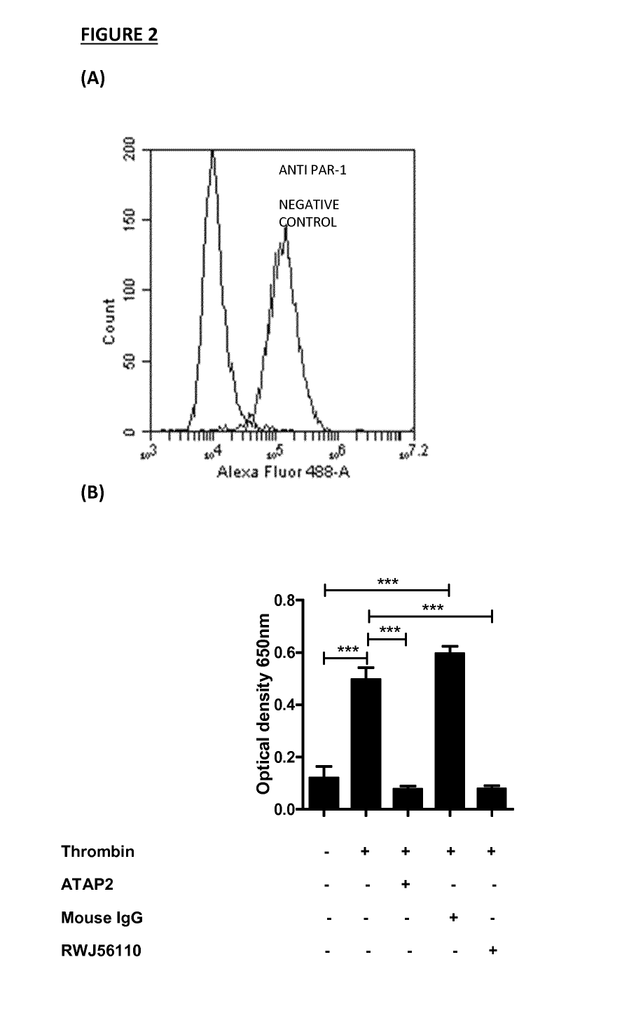

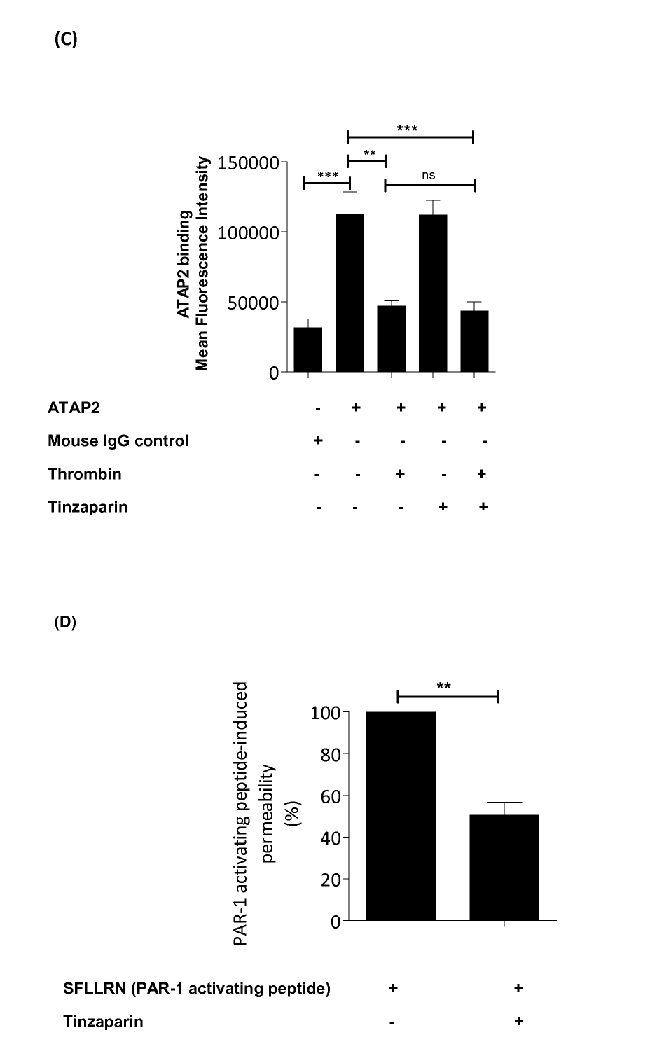

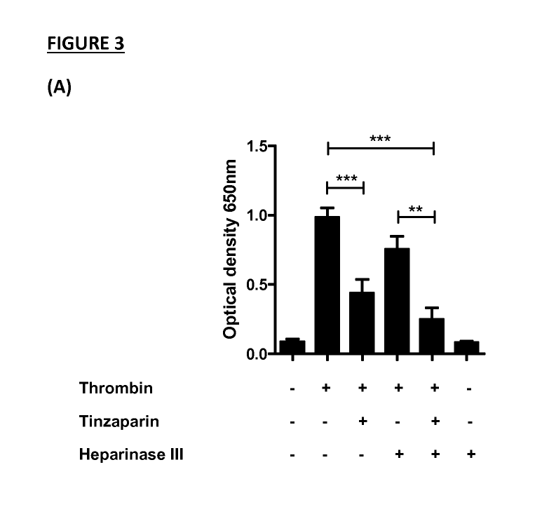

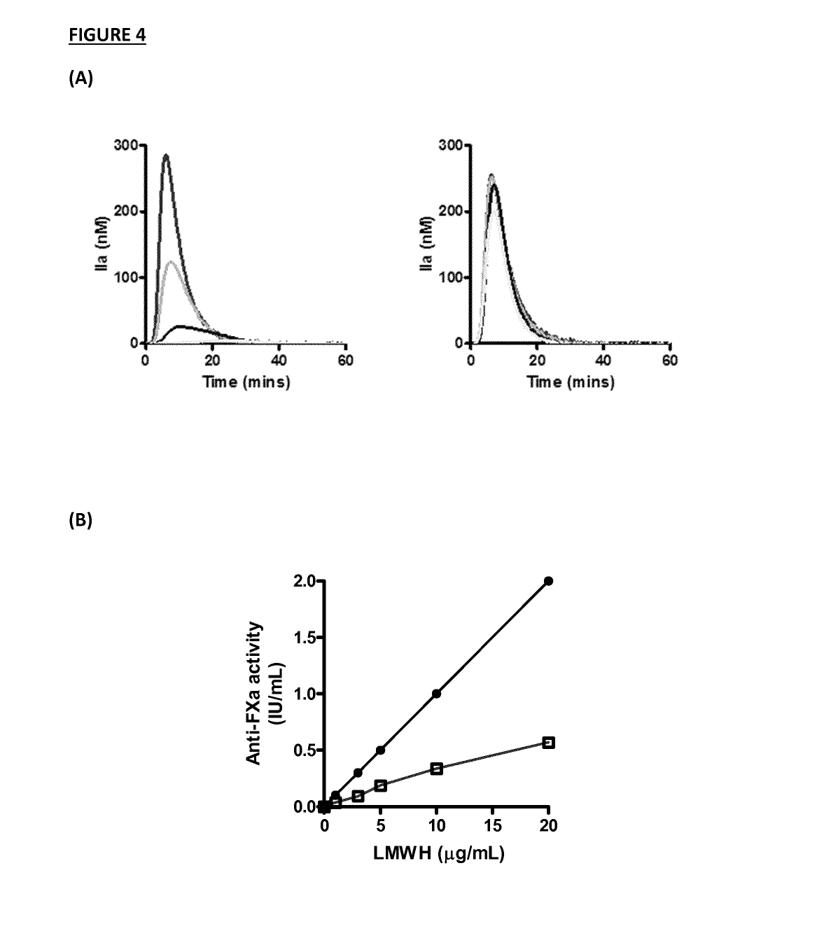

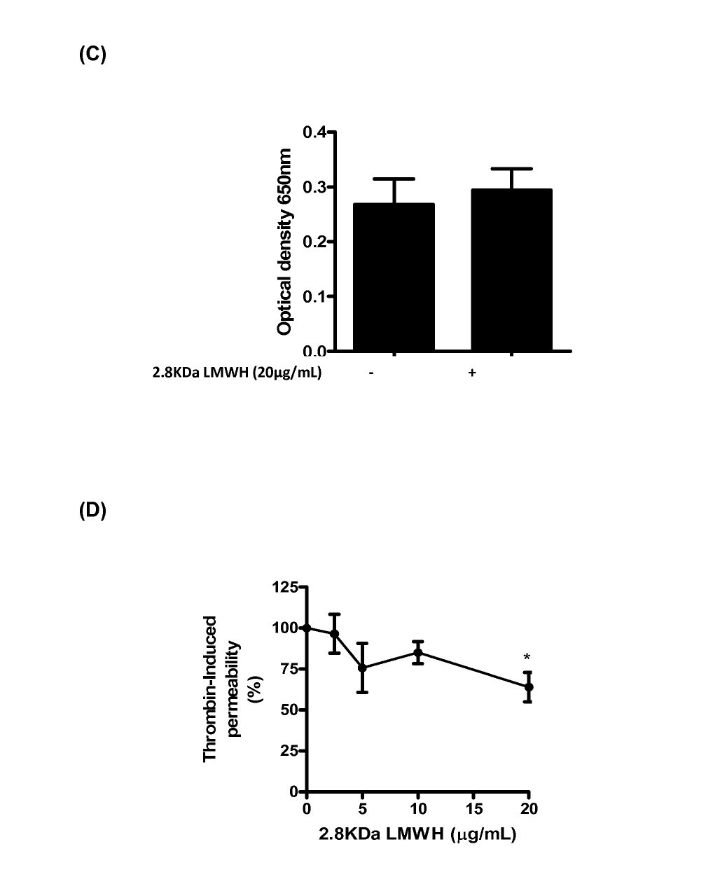

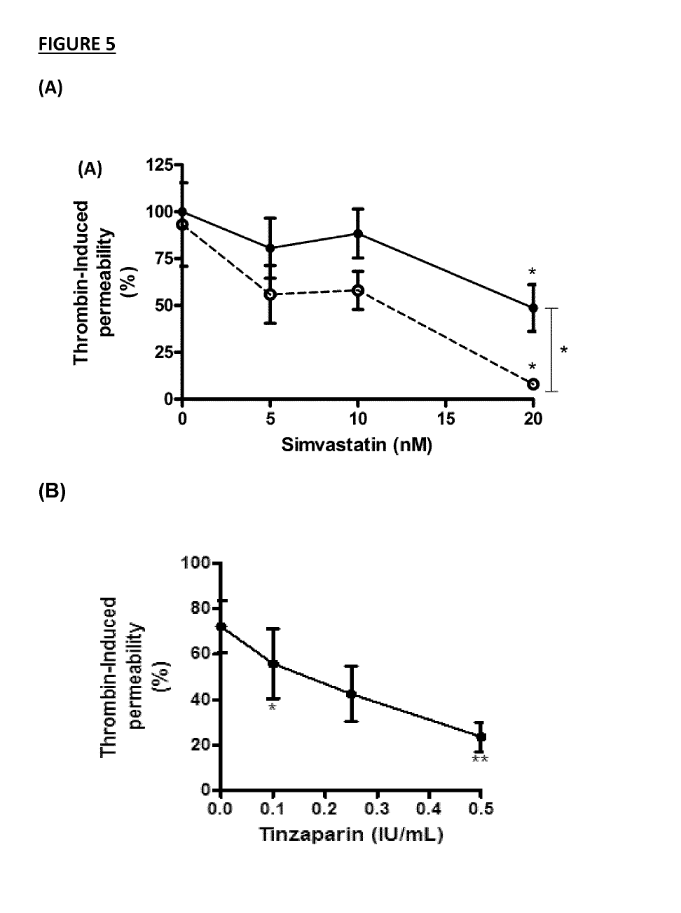

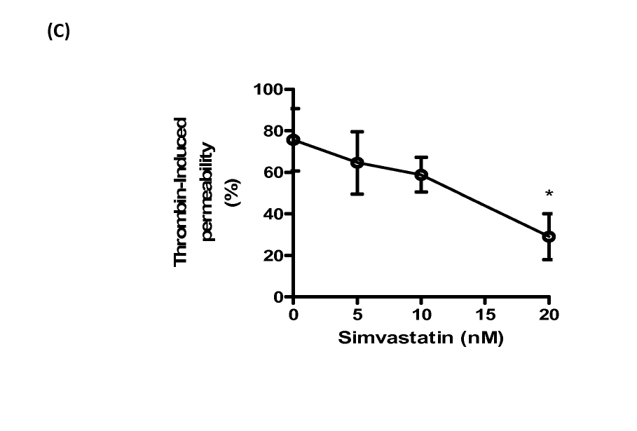

Incubation of confluent monolayers of EA.hy926 endothelial cells with LMWH tinzaparin enhances baseline endothelial barrier function leading to diminished trans-endothelial migration of an Evans blue-conjugated albumin solution (A). Thrombin mediated endothelial barrier dysfunction is characterised by enhanced MLC phosphorylation & actin cytoskeleton activation and loss of inter-endothelial junctional integrity & junctional ZO-1 localisation (B). Thrombin induced loss of monolayer confluence and inter-cellular gap formation is significantly attenuated by tinzaparin (C). Thrombin induces endothelial monolayer permeability (D, left panel) however pre-incubation of endothelial monolayers with tinzaparin leads to a concentration-dependent attenuation of thrombin-induced permeability (D, right panel). Suppression of thrombin-induced endothelial permeability was also observed when endothelial monolayers were incubated with other LMWH formulations (E). LMWH also attenuates VEGF-induced endothelial permeability (F). Experiments were performed at least in duplicate and the presented results represent the mean±SEM of at least three independent experiments (*=p<0.05; **=p<0.01; ***=p<0.001). The presented images are representative of at least three independent experiments.

FIG. 2: LMWH-induced suppression of PAR-1-mediated endothelial barrier dysfunction is not mediated through an inhibition of PAR-1 cleavage and activation

EA.hy926 expression of the PAR-1 receptor was confirmed by flow cytometry (A). Inhibition of PAR-1 cleavage or inhibition of PAR-1 signaling abolishes thrombin-induced endothelial permeability suggesting that thrombin mediated endothelial barrier dysfunction is entirely PAR-1 dependent (B). Tinzaparin does not inhibit PAR-1 expression on endothelial cells and does not inhibit thrombin-mediated PAR-1 cleavage (C). PAR-1 mediated endothelial permeability is attenuated by tinzaparin independent of PAR-1 cleavage (D). Experiments were performed at least in duplicate and the presented results represent the mean±SEM of at least three independent experiments (*=p<0.05; **=p<0.01; ***=p<0.001).

FIG. 3: Cleavage of endothelial cell surface heparan sulphate proteoglycans potentiates the endothelial barrier protective properties of LMWH

Endothelial cell surface HSPG exhibits co-factor activity in supporting thrombin-induced endothelial permeability. HSPG cleavage leads to an increase in LMWH-mediated attenuation of thrombin-induced permeability suggesting that the protective effects of LMWH are not mediated through an interaction with cell surface HSPG (A).

FIG. 4: A 2.8 KDa LMWH fraction derived from tinzaparin exhibits diminished anticoagulant activity in vitro relative to that observed with standard tinzaparin but retains endothelial barrier protective properties

Plasma thrombin generation in pooled normal plasma is suppressed to a greater extent in the presence of tinzaparin (A, left panel; 0 IU/mL, red; 0.1 IU/mL, green; 0.25 IU/mL, blue; 0.5 IU/mL, yellow) relative to that observed in the presence of the 2.8 KDa LMWH fraction (A, right panel; 0 μg/mL, red; 1 μg/mL, green; 2.5 μg/mL, blue; 5 μg/mL, yellow). The 2.8 KDa fraction also exhibits diminished anti-factor Xa activity relative to that observed with equivalent concentrations of standard tinzaparin (B). Incubation of endothelial monolayers with the 2.8 KDa LMWH fraction did not appear to enhance baseline endothelial barrier function (C) but did attenuate thrombin induced endothelial permeability (D). Experiments were performed in duplicate and results are expressed as the mean±SEM of at least three independent experiments (One-way ANOVA with Bonferroni multiple comparisons test; *=p<0.05).

FIG. 5: Simvastatin attenuates endothelial permeability and exhibits synergistic inhibitory effects on agonist-induced endothelial permeability when co-incubated with sub-anticoagulant concentrations of LMWH

Following incubation of endothelial monolayers with simvastatin, at a concentration range similar to the predicted plasma statin concentrations achieved in clinical practice, a modest concentration-dependent attenuation of thrombin-induced endothelial permeability was observed (FIG. 5A; continuous line).

A sub-anticoagulant concentration of tinzaparin (0.1 IU/mL) did not attenuate thrombin-induced endothelial permeability but remarkably following incubation of statin-treated monolayers with tinzaparin at a range of LMWH concentrations, a synergistic effect on endothelial barrier protection was observed and at the sub-anticoagulant tinzaparin concentration of 0.1 IU/mL, the thrombin-induced permeability of EA.hy926 monolayers treated with simvastatin (20 nM) was reduced to just 7.9±0.2% of baseline (p<0.05; FIG. 5A; broken line - - - ).

Similarly, coincubation of endothelial cells with a fixed concentration of simvastatin (20 nM) with a range of LMWH concentrations also resulted in a concentration dependent suppression of endothelial permeability (FIG. 5B).

Co-incubation of endothelial cells with simvastatin at a concentration range similar to the predicted plasma statin concentrations achieved in clinical practice potentiated the endothelial barrier protective properties of the 2.8 KDa LMWH fraction at the non-anticoagulant concentration of 5 μg/mL (0.5 iu/ML) (FIG. 5C).

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, reducing or preventing the incidence of the spread of a primary cancer from one part of the body to a distant locus). In this case, the term is used synonymously with the term “therapy”.

Additionally, the terms “prevention” or “preventing” refers to an intervention (e.g. the administration of an agent to a subject) which prevents or delays the onset or progression of metastatic disease or reduces (or eradicates) its incidence within a treated population. In this case, the term treatment is used synonymously with the term “prophylaxis”.

As used herein the term “sub-anticoagulating heparin formulation” refers to LMWH provided at a sub-anticoagulating dose (i.e. less than 0.25 IU/ml), or a formulation of heparin that is modified to have reduced anticoagulant activity compared with the LMWH Tinzaparin when measured using the anti-coagulant activity assay described below. Anticoagulation activity of heparin can be reduced by depleting the level or activity of the antithrombin-binding pentasaccharide (A-domain). Such modified formulations are referred to herein as non-anticoagulating heparin. Examples of such formulations are described in the literature, for example in Mousa et al (Thromb. Haemost. 2006 December: 96(6) 816-821), Casu et al (Pathophysiol. Haemost. Thromb. 2007 August; 36:195-203), Wang et al (Inflammation Research 2002 (51) Issue 9, 435-443), Ishihara et al (British Journal of Cancer 2002 (86) 1803-1812. The term “sub-anticoagulating heparin formulation” also includes low molecular weight fractions of LMWH, for example fractions having a mean molecular weight of less than 4 or 3 KDa. Examples of such formulations are described herein and in the literature, for example, Dieri et al (Journal of Thrombosis and Haemostasis 2003 (1) 907-914).

As used herein, the term “LMWH” or “low molecular weight heparin” refers to a fraction of heparin that is commonly employed in medicine as a class of anti-coagulant medication. Heparin is a naturally occurring polysaccharide consisting of molecular chains of varying lengths, typically up to 40 KDa. Low molecular weight heparins generally have an average molecular weight of less than 8 KDa and for which at least 60% of polysaccharide chains have a MW of less than 8 KDa. They are obtained by various methods of fractionation and depolymerisation of polymeric heparin. Examples of LMWH on the market include NORMIFLOW, SANDIPARIN, LOVENOX, FLUXUM, INNOHEP, FRAGMIN, CLIVARIN, NADROPARIN. The term also includes fractions or derivatives of LMWH that are depleted in one or more components, for example fractions that are depleted in coagulating components such as the coagulating (antithrombin-binding) pentasaccharide or low molecular weight fractions of LMWH. In one embodiment, the LMWH has a mean molecular weight of 1-8 KDa. In one embodiment, the LMWH has a mean molecular weight of 1-6 KDa or 2-6 KDa. In one embodiment, the LMWH has a mean molecular weight of about 1-3KDa, or 2-3 KDa. In one embodiment, the LMWH has a mean molecular weight of about 3-4 KDa. In one embodiment, the LMWH has a mean molecular weight of about 4-5 KDa. In one embodiment, the LMWH has a mean molecular weight of about 5-6 KDa. In one embodiment, the LMWH has a mean molecular weight of about 6-7 KDa. Method of measuring the mean molecular weight of fractionated heparin are according to the method of Dieri et al. (Journal of Thrombosis and Haemostasis 2003 (1) 907-914).

As used herein, the term “statin” refers to HMG-CoA reductase inhibitors which are a well-known class of lipid lowering drugs indicated for treatment of primary cardiovascular disease and prevention of cardiovascular disease in at-risk patients, especially patients with elevated plasma cholesterol levels. Optionally, the statin component can contain another component, for example a component that promotes statin activity. Exemplary statins include but are not limited to atorvastatin (e.g., LIPITOR), cerivastatin, fluvastatin (e.g., LESCOL), mevastatin, pitavastatin, lovastatin (e.g., MEVACOR or ALTOCOR), provastatin (e.g., PRAVACHOL or SELEKTINE), rosuvastatin (e.g., CRESTOR), and simvastatin (e.g., ZOCOR). The component that promotes statin activity can be a statin stabilizer (e.g., WELCHOL), a fenofibrate (e.g., TRICOR), fish oil (e.g., omega-3), a bile acid sequestrant (e.g., COLESEVELAM), red yeast, Zetia, niacin (e.g., nicotinic acid), or niaspan. Omega-3 can be eicosapentaenoic acid (EPA), docosahexaenoic acid (DHA), or a combination thereof.

As used herein, the term “effective dose or a therapeutically effective dose” as applied to a statin defines an amount that can be administered to a subject without excessive toxicity, irritation, allergic response, or other problem or complication, commensurate with a reasonable benefit/risk ratio, but one that is sufficient to provide the desired effect, e.g. a reduction in endothelial barrier permeability or a reduction in risk of metastasis of a primary cancer in an individual. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes slowing the occurrence or inhibiting the occurrence of metastatic disease, and/or a reduction in endothelial barrier permeability in a treated individual. A therapeutic result need not be a complete cure. In one embodiment, the dose of statin is configured to achieve a statin plasma concentration in the individual of 1-50 nM. In one embodiment, the dose of statin is configured to achieve a statin plasma concentration in the individual of 5-50 nM. In one embodiment, the dose of statin is configured to achieve a statin plasma concentration in the individual of 5-30 nM. In one embodiment, the dose of statin is configured to achieve a statin plasma concentration in the individual of 5-20 nM. In one embodiment, the dose of statin is configured to achieve a statin plasma concentration in the individual of 10-50 nM. In one embodiment, the dose of statin is configured to achieve a statin plasma concentration in the individual of 10-30 nM. In one embodiment, the dose of statin is configured to achieve a statin plasma concentration in the individual of 10-20 nM. Exemplary dosages of a statin may be 5-80 mg, 5-60 mg, 5-50 mg, 5-40 mg, 5-30 mg, 5-20 mg, 5-10 mg, 10-80 mg, 10-60 mg, 10-50 mg, 10-40 mg, 10-30 mg, 10-20 mg. The dosage may be administered once every day, or once every 2, 3, 4, 5, 6, 7 days. The dose may be administered every week, fortnight, month, two months, three months, or four months.

As used herein, the term “sub-anticoagulant” or “sub-anticoagulating” dose of LMHW refers to a dose of LMHW that is administered to a subject which does not cause a clinically effective anti-coagulation effect in the individual. For example, a dosage regime that achieved a plasma concentration of LMHW in an individual of less than 0.3 IU/ml would be considered to be a sub-anticoagulation dose. In one embodiment, the dose is configured to achieve a plasma concentration of LMHW in an individual of less than 0.25 IU/ml. In one embodiment, the dose is configured to achieve a plasma concentration of LMHW in an individual of 0.1 to 0.25 IU/ml. In one embodiment, the dose is configured to achieve a plasma concentration of LMHW in an individual of 0.1 to 0.2 IU/ml. In one embodiment, the dose is configured to achieve a plasma concentration of LMHW in an individual of 0.5 to 0.25 IU/ml. In one embodiment, the dose is configured to achieve a plasma concentration of LMHW in an individual of 0.5 to 0.2 IU/ml. In one embodiment, the dose is configured to achieve a plasma concentration of LMHW in an individual of 0.5 to 0.15 IU/ml. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. The dosage may be administered once every day, or once every 2, 3, 4, 5, 6, 7 days. The dose may be administered every week, fortnight, month, two months, three months, or four months.

In the context of treatment and effective amounts as defined above, the term “individual” (which is to be read to include “subject”, “animal”, “patient” or “mammal” where context permits) defines any individual, particularly a mammalian individual, for whom treatment is indicated. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human.

As used herein, the term “primary cancer” includes all types of cancers that are known to metastasize, including carcinoma (cancers derived from epithelial cells including cancers of the breast, prostate, lung, pancreas and colon), sarcoma (cancers derived from connective tissue such as bone, cartilage, fat and nerve), haematological malignancies (cancers that arise from cells in the blood such as lymphoma and leukaemia), germ cell tumours (cancers derived from pluripotent stem cells that generally present in the testicles and ovaries), and blastoma (cancers derived from immature precursor cells or embryonic tissue that are common in infants and children). The term also includes cancers of unknown primary origin (CUP) and unknown primary tumours (UPT), also referred to as “unknown” or “occult” tumours. Methods of determining whether an individual has a primary cancer and/or is at risk of metastasis are well known to a person skilled in the art and often involve removal of some of the affected tissue (biopsy) and examination/screening of the biopsy for cancer by a pathologist using immunohistochemical stains and visual analysis. In one embodiment, the invention involves screening an individual for metastasis risk factors, and optionally treating the patient according to the invention when one or more risk factors are identified. The risk factors may vary from primary cancer to primary cancer. For example, risk factors for metastasis in primary breast cancer include lymph node involvement, number of cancer-positive lymph nodes, and tumour size. In one embodiment, the individual with a primary cancer has had cancer resection surgery. In one embodiment, the patient with a primary cancer is undergoing treatment for the primary cancer, for example chemotherapy or radiotherapy. In one embodiment, the individual has completed a course of chemotherapy.

As used here, the term “administration” refers to a conventional route of administration to an individual and covers oral or parenteral delivery in any suitable form, e.g., food product, beverage, tablet, capsule, suspension, and solution. The term “parenteral” refers to subcutaneous, intracutaneous, intravenous, intramuscular, intraarticular, intraarterial, intrasynovial, intrasternal, intrathecal, intralesional, and intracranial injection, as well as various infusion techniques. In a preferred embodiment, the statin is administered orally and the sub-anticoagulating heparin formulation is administered parentally, ideally intravenous or by injection. In one embodiment, the sub-anticoagulating heparin formulation and statin are administered together. In one embodiment, the sub-anticoagulating heparin formulation and statin are administered separately. For example, the statin may be administered daily, and the sub-anticoagulating heparin formulation may be administered weekly. In another embodiment, the statin is administered weekly and the sub-anticoagulating heparin formulation is administered monthly.

The sub-anticoagulating heparin formulation and the statin can be formulated separately, or combined together to form a single medicament. Generally, the medicament is a fluid suitable for parenteral administration.

As used herein, the term “low molecular weight fraction of LMWH” means a fraction of heparin or LMWH that exhibits reduced anti-coagulant activity compared with the LMWH Tinzaparin when measured using the anti-coagulant activity assay described below. In one embodiment, the low molecular weight fraction has a mean molecular weight of less than 5 Kda. In one embodiment, the mean molecular weight is less than 4 KDa, or 3 KDa. In one embodiment, the low molecular weight fraction has a mean molecular weight of about 1-4 KDa. In one embodiment, the low molecular weight fraction has a mean molecular weight of about 1-4 KDa. In one embodiment, the low molecular weight fraction has a mean molecular weight of about 1-3 KDa. In one embodiment, the low molecular weight fraction has a mean molecular weight of about 2-4 KDa. In one embodiment, the low molecular weight fraction has a mean molecular weight of about 2-3 KDa.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Experimental

### Materials

EA.hy926 cells (an immortalised, PAR-1 expressing human umbilical vein endothelial cell line) were a kind gift from Dr C. Edgell (University of North Carolina Chapel Hill, USA). Polyethylene terephthalate (PET) membrane trans-well inserts (Millicell® Hanging Cell Culture Inserts, 3.0 μm pore size, 4.5 cm2 membrane surface area) were purchased from Merck Millipore Corporation (Massachusetts, USA). LMWH tinzaparin and a 2.8 KDa tinzaparin-derived LMWH fraction were from Leo Pharma® (Ballerup, Denmark). LMWH enoxaparin and unfractionated heparin were from Sanofi® (Paris, France) and Wockhardt® (Wrexham, United Kingdom) respectively. Dulbecco's modified eagle medium (DMEM), fetal bovine serum (FBS), penicillin-streptomycin, hypoxanthine-aminopterin-thymidine (HAT supplement), AlexaFluor488® Phalloidin, Rabbit anti-ZO-1 primary antibody, AlexaFluor488® goat anti-rabbit secondary antibody and AlexaFluor488® donkey anti-rabbit secondary antibody were purchased from Invitrogen® (California, USA). RWJ56110, a selective PAR-1 antagonist was from R&D Systems Inc. (Minnesota, USA). Rabbit anti phospho-MLC-2 (Thr18/Ser19) primary antibody was purchased from Cell Signaling Technology® (Massachusetts, USA). Human alpha-thrombin was purchased from Haematologic technologies® (Vermont, USA). Thrombin generation reagents (thrombin calibrator, PPP-reagent, FluCa thrombin substrate) were purchased from Thrombinoscope® BV (Maastricht, The Netherlands). HaemosIL® Liquid anti-Xa reagent and HaemosIL® pooled normal control plasma were from Instrumentation Laboratory (Minnesota, USA). Recombinant human VEGF165, simvastatin, anti-PAR-1 receptor antibody (ATAP2), PAR-1 activating peptide (Ser-Phe-Leu-Leu-Arg-Asn-amide; SFLLRN), heparinase III (from flavobacterium Heparinum) and all other chemicals and reagents, unless otherwise stated, were purchased from Sigma Aldrich® (Missouri, USA).

### Endothelial Barrier Permeability Assay

An in vitro assay of endothelial barrier function was established as previously described. Briefly, EA.hy926 human endothelial cells were seeded at a concentration of 20×104 on PET membrane inserts in the apical chamber of trans-well cell culture plate inserts and cultured in DMEM supplemented with 10% FBS for 72 hours at 37° C. in a humidified atmosphere of 5% carbon dioxide in air. At 72 hours, the confluent EA.hy926 monolayers formed on the PET membranes were washed in warmed sterile phosphate buffered saline (PBS), and re-incubated in serum-free DMEM. An Evans Blue (0.67 mg/mL)-conjugated bovine serum albumin (BSA, 4%) solution was added to the apical chamber of each trans-well and aliquots of cell culture media from the basolateral chamber of the trans-well plate were then sampled at 2 minute intervals. Using a plate reader (SpectraMax®M2 Microplate Reader; Molecular Devices LLC, California, USA) in conjunction with SoftMax Pro® software (Version5.4.1; Molecular Devices LLC, California, USA) the permeability of the endothelial cell layers was determined through the spectrophotometric measurement of the increase in absorbance in the sampled media as a result of transmigration of the Evans Blue-BSA solution through the endothelial cell layer over time.

### Characterisation of Endothelial Cell F-actin and ZO-1 Localisation

EA.hy926 cell monolayers grown on glass coverslips were fixed in 3.7% paraformaldehyde and permeabilised in triton-X-100 (0.1% solution in PBS). In order to characterise the pattern of distribution of F-actin and the extent of F-actin stress fibre formation in endothelial cells under basal conditions, the fixed monolayers were first blocked in a 5% BSA solution for 60 mins, washed and incubated with AlexaFlour488®phalloidin (a probe directed against F-actin) for 30 minutes in darkness at room temperature. The coverslips were then mounted on glass slides and the EA.hy926 cells visualised by immunofluorescence microscopy (Axioplan 2 imaging® fluorescence microscope; Carl Zeiss AG, Gottingen, Germany) in conjunction with Axiovision software (Version 4.2.8; Carl Zeiss AG, Gottingen, Germany).

The pattern of distribution of ZO-1 was determined by first incubating fixed coverslips in a 5% donkey serum blocking solution following which a 1:200 dilution of a rabbit anti-ZO-1 primary antibody was applied and the coverslips incubated at 4° C. overnight. The coverslips were then washed in PBS and incubated for 60 minutes in darkness at room temperature with a goat-anti-rabbit IgG fluorescent secondary antibody (AlexaFlour488® Goat-anti-rabbit). The coverslips were then mounted on glass slides and imaged as outlined above.

Characterisation of Endothelial Cell Myosin light Chain-2 Phosphorylation

EA.hy926 cells were grown to 80% confluence on glass coverslips following which they were fixed, permeabilised and blocked in a 5% donkey serum blocking solution as outlined above. A 1:200 dilution of a rabbit anti-phospho-MLC-2 (Thr18/Ser19) antibody was then applied and the coverslips were incubated overnight at 4° C. The endothelial cell layers were then washed in PBS and incubated with 1:100 dilution of AlexaFluor488 donkey-anti-rabbit IgG secondary antibody in PBS for 1 hour in darkness at room temperature. The coverslips were then mounted on glass slides and the pattern of MLC diphosphorylation visualised by immunofluorescence microscopy.

### Confirmation of PAR-1 Expression on the Endothelial Cell Surface

EA.hy926 endothelial cells were suspended in a 5% goat serum solution and incubated with the ATAP2 anti-PAR-1 receptor antibody or a mouse IgG control for 60 minutes on ice. The EA.hy926 cells were then re-suspended in a 1:1000 dilution of AlexaFluor488 goat anti-mouse IgG for 30 minutes on ice and the expression of PAR-1 determined by the measurement of the cellular fluorescence intensity by flow cytometry.

### Cleavage of Endothelial Cell Surface Heparan Sulphate Proteoglycans (HSPG)

Monolayers of EA.hy926 endothelial cells grown on PET membrane trans-well inserts were incubated with heparinase III (1 unit/mL) for 2 hours at 37° C. The cell culture supernatant was aspirated and the monolayers washed three times with warmed, sterile PBS and re-incubated with fresh cell culture media. The heparan sulphate composition of the aspirated supernatant was determined by mass spectrometry.

### Assessment of Parameters of Plasma Thrombin Generation and Plasma Anti-FXa Activity

Plasma thrombin generation was assessed by calibrated automated thrombography using a Fluoroskan Ascent® Plate Reader (ThermoLab Systems®, Helisinki, Finland) in conjunction with Thrombinoscope™ software (Thrombinoscope BV, Maastricht, The Netherlands) as previously described.

Briefly, 80p1 aliquots of normal pooled plasma were incubated with 20 μL of platelet-poor-plasma reagent (PPP-Low reagent) containing 1 pM TF and 4 μM phospholipids (composed of 60% phosphatidylcholine, 20% phosphatidylserine, and 20% phosphatidylethanolamine) in a 96-well round-bottom polystyrene plate (Nunc™ microwell plates; ThermoScientific™, Massachusetts, USA). Thrombin generation was then initiated by the automatic dispensation of a fluorogenic thrombin substrate (Z-Gly-Gly-Arg-Amido-4-methylcoumarin hydrochloride) and 100 mM CaCl2 into each well (final concentrations, Z-Gly-Gly-Arg-AMC.HCl, 0.42 mM and CaCl2, 16.67 mM) and assessment of thrombin generation parameters was determined using a thrombin generation standard. The lagtime to initiation of thrombin generation, peak thrombin generation, time to peak thrombin generation and the area under the thrombin generation curve (endogenous thrombin potential; ETP) was determined for each plasma sample.

Plasma anti-factor Xa activity was measured using ACL TOP® 500 haematology analyser (Instrumentation Laboratory, Minnesota, USA) in conjunction with the HaemosIL® liquid Anti-Xa reagent.

### Low Molecular Weight Fractions of LMWH

Low molecular weight LMWH fractions of lower molecular weight are isolated from standard LMWH preparations by filtration through a column of Sephadex G-100 equilibrated with 0.15 M NaCl in 0.01 M Tris-HCl, pH 7.5 [Salzman, E. W., et al., Effect of heparin and heparin fractions on platelet aggregation. J Clin. Invest, 1980. 65(1): p. 64-73.]. LMWH fractions of low molecular weight have significantly attenuated anticoagulant function compared with standard LMWH but retain their anti-coagulant activity (FIG. 4).

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A combination of drugs for use in a method for the prevention or treatment of cancer metastasis in an individual with a primary cancer, in which the combination is administered to the individual and comprises a sub-anticoagulating heparin formulation and a therapeutically effective dose of a statin.

2. A combination of drugs of claim 1, for use of claim 1, in which the sub-anticoagulating heparin formulation comprises a sub-anticoagulation dose of low molecular weight heparin (LMWH).

3. A combination of drugs of claim 1 or 2, for use of claim 1, in which the sub-coagulating dose of LMWH is sufficient to achieve a plasma concentration of LMWH in the individual of less than 0.25 IU/ml.

4. A combination of drugs of claim 1 or 2, for use of claim 1, in which the sub-anti-coagulating dose of LMWH is sufficient to achieve a plasma concentration of LMWH in the individual of about 0.05 to 0.2 IU/ml.

5. A combination of drugs of claim 1, 2 or 3, for use of claim 1, in which the therapeutically effective dose of statin is sufficient to achieve a plasma concentration of statin in the individual of 5-30 nM.

6. A combination of drugs of any of claims 1 to 5, for use of claim 1, in which the sub-anticoagulating heparin formulation is administered parenterally and the statin is administered orally.

7. A combination of drugs of any of claims 1 to 5, for use of claim 1, in which the primary cancer is an early stage cancer.

8. A combination of drugs of any of claims 1 to 5, for use of claim 1 or 7, in which the individual does not have elevated plasma cholesterol levels.

9. A combination of drugs of any of claims 1 to 5, for use of claim 1,7 or 8, in which the LMWH formulation is a low molecular weight heparin formulation.

10. A combination of drugs of any of claims 1 to 5, for use of claim 1, 7 or 8, in which the sub-anticoagulating heparin formulation is administered after administration of the statin.

11. A pharmaceutical composition comprising a sub-anticoagulating heparin formulation, a therapeutically effective statin, and a pharmaceutically acceptable excipient.

12. A pharmaceutical formulation as claimed in claim 11 in which the sub-anticoagulating heparin formulation comprises a sub-anticoagulating dose of LMWH.

13. A pharmaceutical composition as claimed in claim 11 or 12 in the form of a liquid suitable for parenteral administration to an individual.

14. A kit of parts comprising one or more doses of a sub-anticoagulating heparin formulation and one or more therapeutically effective doses of a statin.

15. A kit of parts according to claim 14, in which the sub-anticoagulating heparin formulation is formulated as a liquid suitable for parenteral administration and the statin is formulated for oral administration.

16. A low molecular weight fraction of LMWH, for use in a method for the prevention or treatment of cancer metastasis in an individual with a primary cancer.

17. A low molecular weight fraction of LMWH of claim 16, for use of claim 16, in which the low molecular weight fraction has a mean molecular weight of less than 5 KDa.

18. A low molecular weight fraction of LMWH of claim 16, for use of claim 16, in which the low molecular weight fraction has a mean molecular weight of less than 4 KDa.

19. A low molecular weight fraction of LMWH of claim 16, for use of claim 16, in which the low molecular weight fraction has a mean molecular weight of less than 3 KDa.

20. A low molecular weight fraction of LMWH of claim 16, for use of claim 16, in which the low molecular weight fraction has a mean molecular weight of about 2-4 KDa.

21. A low molecular weight fraction of LMWH of claim 16, for use of claim 16, in which the low molecular weight fraction has a mean molecular weight of about 2-3 KDa.
